(19) United States
(12) Patent Application Publication
(10) Pub. No.: US 2005/0265947 A1
(43) Pub. Date: Dec. 1, 2005

(54) Method for thickening hair and hair thickener

(76) Inventor: Sanford E. Schnoll, San Francisco, CA (US)

Correspondence Address: SCHNECK & SCHNECK, P.O. BOX 2-E, SAN JOSE, CA, 95109-0005, US

(21) Appl. No.: 10/955,785

(22) Filed: Sep. 29, 2004

Related U.S. Application Data
(63) Continuation-in-part of application No. 10/854,692, filed on May 25, 2004.

(51) Int. Cl. A61K 7/06; A61K 7/11

(52) U.S. Cl. 424/70.14

(57) ABSTRACT

Particles of hair, each particle having no dimension greater than 3 mm, are prepared and affixed to a desired region, which may or may not have hair, to achieve thickening of the hair on the region or to add hair to the region, if lacking in hair. Such regions include the scalp or eyebrow. The particles of hair may be prepared by freezing and then pulverizing, chopping, or milling the frozen hair into the desired size. Alternatively, the hair is not frozen during preparation of hair particles. The hair particles may be combined with a carrier, such as a hair styling agent, for affixing.

### CROSS-REFERENCE TO RELATED APPLICATION

This application is a continuation-in-part of and claims the benefit of priority from U.S. patent application Ser. No. 10/854,692, entitled “Method for Thickening Hair and Hair Thickener”, which is hereby incorporated by reference.

### FIELD OF THE INVENTION

The present invention relates to hair thickener and methods for preparing and affixing hair thickeners.

### BACKGROUND OF THE INVENTION

Hair loss is a common problem for many men and women. As a consequence, systems and methods to supplement regions of thinning hair have been developed.

In some systems and methods, synthetic fibers are utilized to supplement regions of thinning hair. U.S. Pat. No. 5,005,596 to Yamada, discloses artificial hair made of a synthetic filament with silver particles applied to the filament by vacuum deposition. The artificial hair is used for hair implantation.

In other systems and methods, fibers comprised of the same organic keratin protein as natural hair are sprayed onto a scalp to supplement regions of thinning hair. Animal fibers, such as ground-up sheep's wool, are utilized.

In additional systems and methods, natural human hair may be utilized to supplement thinning hair. U.S. Pat. No. 5,575,298 to Hinton discloses use of human hair particles on a layer to join a hair supplement to the scalp. The layer includes an adhesive on a lower surface of the layer which is used for attachment to human hair on the scalp.

Natural human hair is a complex structure comprised of a hair shaft including a cortex, a cuticle, microfibrils and macrofibrils. Microfibrils and macrofibrils are composed of keratin. Use of natural human hair is desirable, as it typically appears more natural looking than synthetic hairs, keratin protein alone, or animal hairs. Though keratin protein or animal hairs may have a protein in common with natural human hair, they are lacking in remaining structural elements of natural human hair.

It is an object of the present invention to provide a method and composition for increasing the thickness of hair.

It is another object of the present invention to increase thickness of hair.

It is an additional object of the present invention to provide a method and composition concealing hair loss.

### SUMMARY OF THE INVENTION

These and other objects have been achieved by preparing a composition of particles of natural human hair, each of the particles ranging in size from 3 mm or less, and affixing the human hair particles to a desired region, for example, human hair located on a region of the human body, such as a scalp or an eyebrow. Additionally, or alternatively, the particles adhere to a surface lacking in hair, including a scalp. Because the particles are comprised of natural human hair, the particles blend in naturally with the existing hair while thickening a person's hair. Further, the small size of the particles are advantageous because larger particles do not appear as natural or tend to fall off the person. The small size of the particles allows a person to gradually build on the thickness of adhered particles and/or the hair to be supplemented resulting in a natural looking, desired amount of hair. The user may utilize particles of approximately the same size or of varied sizes to build on the thickness of existing hair to a desired level and/or to build the thickness of the particles to a desired level. The human hair particles may be comprised of a desired type or color.

The human hair particles, in one embodiment, are prepared by cutting larger dimensioned hair into the smaller specified size of 3 mm or less. Larger dimensioned hair is typically greater than 3 mm in size.

In another embodiment, the particles are prepared by snap freezing larger dimensioned human hair and by pulverizing the frozen hair into the specified size at 3 mm or less. Pulverizing includes one or more methods for reducing the size of the larger dimensioned hair such as crushing or grinding, as well as other methods. The particles may be in powder or granular form.

In another embodiment, larger dimensioned human hair is frozen and placed in a blender for processing. The blender may include a whirling blade that chops the frozen hair into hair particles of the specified size of 3 mm or less for use as a hair thickener.

In another embodiment a mill, such as an ultra-centrifugal mill, is used to mill larger dimensioned hair into hair particles of the specified size of 3 mm or less for use as a hair thickener.

Fixing agents may be applied to the human hair particles to affix the hair particle to the desired location. In one example, the human hair particles are combined with a carrier, such as a hair styling agent for affixation.

Also, the human hair particles may be adhered to hair supplements, such as hair pieces, to thicken the appearance of the hair on the hair pieces. The hair pieces may be comprised of, for example, synthetic hair.

In one embodiment, animal hair may be used as the hair thickener of the present invention. Larger dimensioned animal hair, instead of, or in addition to, larger dimensioned human hair, may be used in methods of preparing the hair particles of the specified size or 3 mm or less as described herein.

### BRIEF DESCRIPTION OF THE DRAWINGS

FIG. 1 is a perspective view of hair particles of the present invention.

FIG. 2 is a perspective view of hair, on a portion of a surface, prior to attachment of the hair particles of FIG. 1.

FIG. 3 is a perspective view of hair particles of FIG. 1 attached to the hair and surface of FIG. 2.

### DETAILED DESCRIPTION OF THE INVENTION

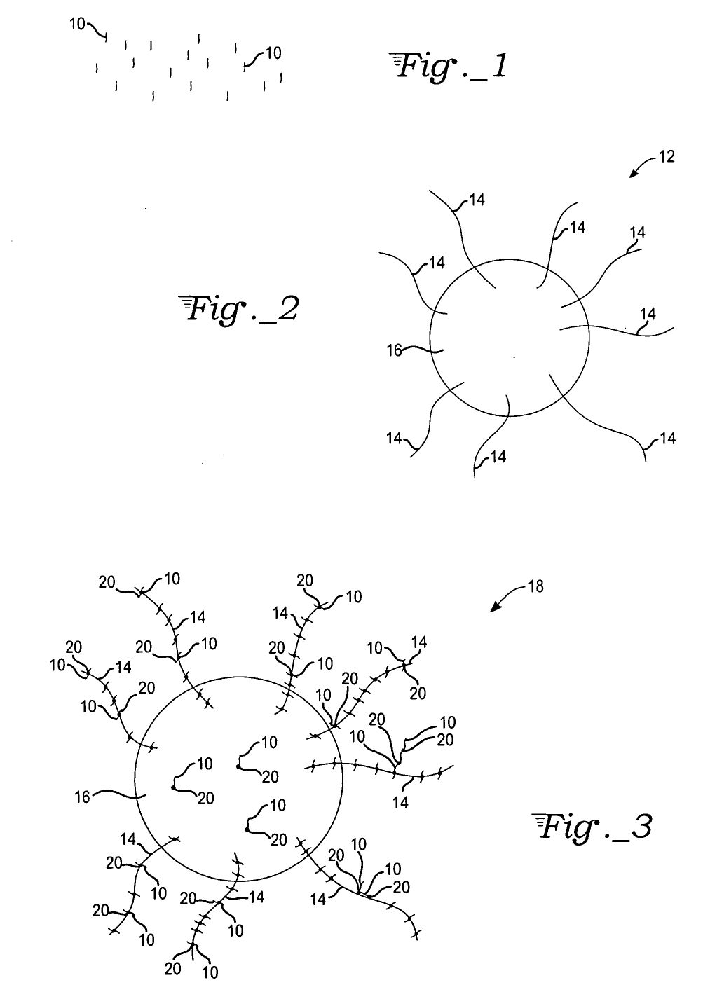

FIG. 1 illustrates hair particles 10 of the present invention. The hair particles 10 of the present invention range in size from 3 mm or less. In one embodiment of the present invention, the hair particles 10 are human hair particles. In another embodiment, the hair particles 10 are animal hair particles 10, such as sheep's wool. The methods of producing the hair particles 10 and uses for the hair particles 10 are described below with regard to human hair. However, animal hair may be substituted for or supplement human hair in methods and uses described below, if desired. The particles 10 may be small enough to comprise a powder or to be granular in form. Typically, no dimension of the hair particles 10 is greater than 3 mm. For example, where the particles 10 are round, no diameter is greater than 3 mm and where the particles are straight, as depicted, no length or width of the particle 10 is greater than 3 mm. Though only one shape and type of particles 10 is illustrated, the particles 10 may vary in shape and type and may include a desired mixture of shapes and types. Hair types include for, example, curly or straight. Further, the hair particles 10 are comprised of a desired color or mixture of colors.

In one example, a method for harvesting human hair particles 10 comprises cutting human hair strands/pieces to a particle size of 3 mm or less as described above. Scissors or other equipment may be used to cut hair in to the desired particle size. The human hair to be cut (not shown) may be obtained from the person whose hair is to be thickened or it may be obtained from other sources, such as businesses that specialize in the sale of human hair.

In another method for harvesting human hair particles 10, human hair strands are frozen. For example, the human hair may be snap frozen in liquid nitrogen. In another example, the human hair is frozen with dry ice powder. The human hair to be frozen (not shown) may be obtained from the person whose hair is to be thickened or it may be obtained from other sources. The frozen human hair is pulverized, or otherwise processed, into human hair particles 10 of the present invention.

In one example of preparing particles 10, hair is cut into pieces approximately 5 mm in length. The cut hair pieces are mixed with liquid nitrogen at, for example, −196 degrees Celsius. The frozen hair is then pulverized into particles 10 of the present invention. In one example, pulverizing the frozen hair includes crushing the hair with a mortar and pestle. In another example, pulverizing includes crushing the frozen hair with a mechanical mortar and pestle. In another example, pulverizing includes grinding.

Once the hair is pulverized, cut, or otherwise processed into particles 10 of the present invention, the particles may be separated by size. A sizing sieve may be utilized to separate by size. Particles 10 may be divided into particles of the same size or of different sizes, depending on the user's or manufacturer's preference.

In one embodiment of the present invention, hair particles 10 are harvested by combining human hair strands of a size, typically larger than 3 mm in length, with liquid nitrogen in a blender (not shown). In one example, an amount of liquid nitrogen is added to cover the hair present within the blender. The liquid nitrogen freezes the hair. The blender is then activated to process the liquid nitrogen and hair mixture resulting in hair particles 10 of the present invention, of a size as described above. The blender includes, for example, a whirling blade. The blade may rotate to chop the hair in the mixture into the particles 10. The blender may process the hair in other ways using other functions present on a blender. In one example, the blender is an industrial blender. In another example, the blender is a Waring® blender. The hair particles 10 may be separated by size as described above with, for example, a sizing sieve.

In another embodiment of the present invention, hair particles 10 are harvested with the use of a mill, such as an ultra-centrifugal mill (not shown) or a planetary ball mill (not shown). An example of an ultra-centrifugal mill used, is the Retsch® Ultra-Centrifugal Mill ZM 200. Human hair to be processed (not shown) is cut into strands that are small enough to be milled by the ultra-centrifugal mill, if the hair is not already of a size that can be milled. In one example, the hair is cut into strands that are about 2 cm in length or less to facilitate milling. The hair is cut, for example, by hand with scissors, or with a knife mill such as a Glenmill® knife mill at, for example, room temperature. The cut hair or hair to be milled is mixed with dry ice, such as dry ice powder, and the hair and dry ice mixture is placed in the mill such as the Retsch® ultra-centrifugal mill for milling the hair into particles 10. The ultra-centrifugal mill includes a vibratory feeder which vibrates and allows for continuous automated feeding of the dry ice/hair mixture. For example, during milling, a mill rotor sheers the hair into hair particles 10. Hair particles 10, which may be in a powder form, result from the milling.

In one example, human hair strands that are about, for example, 2 cm or less in length, are placed in a mill such as an ultra-centrifugal mill for milling, without dry ice. The hair particles 10 that result may have a burnt or bad smell. Therefore, the hair particle 10 may be washed, aired out, or have a fragrance applied to them to remove or mask the smell. Airing out the particles includes exposing the particles to air before use. The hair particles 10 may be divided by size with, for example, a sizing sieve.

An example of a planetary ball mill used is the Retsch® Planetary Ball Mill, Type PM 400. Human hair to be processed is cut into strands that are small enough to be milled by the planetary ball mill, if the hair is not already of a size that can be milled. The hair may or may not be frozen during or before milling. In one example, hair is snap frozen to create smaller hair pieces. Which are further reduced in size by the planetary ball mill. Hair to be processed, frozen or non-frozen, is placed in the planetary ball mill for milling. The hair is milled into hair particles 10. Milling, includes, for example, pulverizing or grinding.

Milling hair allows for production of hair particles 10 that are, for example, a powder. The hair particles 10 that are produced by milling are for example, 2 mm or less in length. Typically, the majority of particles produced by milling are less than 1 mm in length.

With regard to all of the processes described herein to produce human hair particles 10, it is important to note that the human hair particles 10 still retain and/or contain the entire complex structure of human hair including a hair shaft with a cortex, a cuticle, microfibrils, and macrofabrils. In other words, the human hair particles 10 typically include either individual particles that include the entire complex structure of human hair and/or a mixture of particles 10 that contains all of the structural elements of hair within the mixture.

A region 12, which is desired to be supplemented with hair particles 10 made by, for example, the methods of the present invention as described herein, includes hair 14 to be supplemented and a surface 16, as illustrated in FIG. 2. In one example, the surface 16 is a body surface and the hair 14 which is desired to be supplemented with hair particles 10 comprises human hair on the body surface 16. For example, the surface 16 may comprise a human scalp and the hairs 14 to be supplemented may comprise strands of human hairs on the scalp. In another example, the surface 16 may comprise a brow region of a head and the hairs 14 to be supplemented in thickness may include hairs of an eyebrow on the brow region.

In another example, the hair 14 which is desired to be supplemented is a part of a hair piece, such as a toupee or wig. The hair piece may include artificial hair (synthetic or animal hair) and the surface 16 may be a synthetic surface.

A region 18 supplemented with hair particles 10 is illustrated in FIG. 3. The human hair particles 10 of the present invention may be affixed to the hair 14 to be supplemented or the desired surface 16 resulting in a thickening of the hair 14. Further, the human hair particles 10 may be affixed to other particles 10. A fixing agent 20 is depicted in FIG. 3, affixing the human hair particles 10 to the hair 14 to be supplemented, to hair particles 10 which have been affixed to human hair particles 10, to hair particles 10 which have been affixed to the hair 14, and to the surface 16.

The small size of the particles 10 of the present invention allow a person to gradually build on the thickness of the hair 14 to be supplemented resulting in a natural looking, desired amount of hair. Further, the particles 10 may build on other particles 10 which have already been affixed to the hair 14 to be supplemented or to the surface 16 for a natural look. The user may utilize particles 10 of approximately the same size or of a varied size to build on the thickness of existing hair 14 or particles 10 to a desired level. Also, the particles 10 conceal regions lacking in hair. Whether of a varied size or not, the particles 10 are less than 3 mm in dimension, as described above.

The user may use hair particles 10 which are the same color as the hair 14 to be supplemented, or the user may use hair particles 10 which are different in color from the hair 14 to be supplemented in order to change the color of the hair 14 to be supplemented.

The hair particles 10 of the present invention are affixed to a desired surface, to human hair disposed on the body surface, to other particles 10, and to hair on synthetic surfaces in various ways. A fixing agent 20 is used to affix the hair particles 10 to the hair 14 to be supplemented, to hair particles 10, and/or to the surface 16. For example, the hair particles 10 of the present invention are dispersed within a carrier, such as a hair styling product and the carrier, including the particles 10, is applied to the hair 14 to be supplemented. Hair styling products include, for example, mousse, cream, gel, pomade, and spray. The carrier acts as the fixing agent 20 or adhesive. In one example, hairspray or other fixative may be applied in addition to the fixing agent 20.

In another example, the human hair particles 10 are applied to the hair 14 to be supplemented, the human hair particles 10 which have already been affixed to hair 14 or surface 16, and/or to the desired surface 16, and are affixed with the fixing agent 20, such as hairspray. The hairspray is sprayed onto the human hair particles 10, hair 14, and/or surface 16 to affix the human hair particles 10. Hairspray may be sprayed onto human hair particles 10 of the present invention, onto hair 14, or onto surface 16 after the particles have been applied to hair 14 which is desired to be thickened, to affixed particles 10, and/or to surface 16. Hairspray acts as the fixing agent 20 or adhesive which affixes the human hair particles 10 of the present invention to the surface 16, hair 14, and/or particles 10 on the surface 16.

In one example, the particles 10 may be applied to surface 16, hair 14, and/or particles 10 on the surface 16 without a fixing agent.

The drawings are for illustrative purposes only and are not intended to limit the invention. In particular, the drawings are not intended to limit the location, orientation, or amount of the fixing agent 20 or the hair particles 10 of the invention.

1. A method for thickening human hair:
freezing hair strands;
harvesting smaller particles of hair from frozen hair strands; and
applying said particles of hair to human hair, thereby thickening human hair.

2. The method of claim 1 wherein said human hair is affixed to a person.

3. The method of claim 2 further comprising affixing said particles of hair to human hair affixed to said person.

4. The method of claim 1 wherein each of said particles range in dimension from 3 mm or less.

5. The method of claim 1 wherein said particles of hair are powder.

6. The method of claim 1 wherein said particles of hair are human hair.

7. The method of claim 1 wherein said particles of hair are animal hair.

8. The method of claim 1 wherein freezing said hair strands includes freezing said hair strands in liquid nitrogen, and further comprising pulverizing said hair strands into said smaller particles of hair.

9. The method of claim 8 wherein pulverizing includes grinding.

10. The method of claim 8 wherein pulverizing includes crushing.

11. The method of claim 1 wherein freezing said hair strands includes freezing said hair in liquid nitrogen.

12. The method of claim 11 wherein said hair strands are cut into smaller strands prior to freezing hair.

13. The method of claim 12 wherein said cut strands are about 5 mm in length.

14. The method of claim 12 wherein said harvesting occurs by pulverizing said hair strands after freezing said hair strands.

15. The method of claim 14 wherein pulverizing includes grinding.

16. The method of claim 14 wherein pulverizing includes crushing.

17. The method of claim 1 wherein said freezing includes combining said hair strands with liquid nitrogen.

18. The method of claim 17 further comprising processing said hair strands and liquid nitrogen combination with a blender.

19. The method of claim 18 wherein said processing includes chopping said frozen hair strands with a rotating blade of said blender.

20. The method of claim 1 wherein said harvesting occurs by chopping said frozen hair strands with a rotating blade.

21. The method of claim 1 wherein said freezing includes mixing said hair strands with dry ice.

22. The method of claim 21 further comprising milling said hair strands and dry ice mixture in a mill.

23. The method of claim 22 wherein said mill is an ultra-centrifugal mill.

24. The method of claim 23 wherein said mill is Retsch® ultra-centrifugal mill.

25. The method of claim 22 wherein milling includes shearing.

26. The method of claim 1 wherein harvesting said smaller particles of hair includes milling said frozen hair strands.

27. The method of claim 26 wherein said milling occurs with an ultra-centrifugal mill.

28. The method of claim 26 wherein said milling occurs with a planetary ball mill.

29. The method of claim 23 wherein said hair strands mixed with said dry ice are 2 cm or less in length.

30. The method of claim 23 wherein said particles of hair range in length from 2 mm or less.

31. The method of claim 30 wherein said particles of hair are 1 mm or less in length.

32. The method of claim 22 wherein said particles of hair range in length from 2 mm or less.

33. The method of claim 23 wherein said hair particles are a powder.

34. The method of claim 23 wherein said hair particles include an entire structure of hair.

35. The method of claim 1 further comprising separating said hair particles by size.

36. A method for thickening human hair affixed to a person, comprising:
freezing hair strands of a relatively large dimension;
harvesting relatively smaller particles of hair from frozen hair strands; and
affixing said relatively smaller particles of hair to human hair affixed to a person, thereby thickening human hair.

37. The method of claim 36 wherein said harvesting occurs by pulverizing.

38. The method of claim 36 wherein said harvesting occurs by chopping.

39. The method of claim 36 wherein said harvesting occurs by milling.

40. A hair product for thickening hair, comprising:
particles of hair having no dimension greater than 3 mm, said particles previously frozen; and
a carrier within which said particles of hair are dispersed.

41. The hair product of claim 40 wherein said carrier is a hair styling product.

42. The hair product of claim 40 wherein said carrier is a fixative.

43. The hair product of claim 40 wherein said particles of hair are powder.

44. The hair product of claim 40 wherein said particles of hair are granular.

45. The hair product of claim 40 wherein said particles of hair are animal hair.

46. The hair product of claim 40 wherein said particles of hair are human hair.

47. The hair product of claim 40 wherein individual particles of hair include an entire structure of hair.

48. The hair product of claim 40 wherein said particles of hair comprise a mixture containing all elements of an entire structure of hair.

49. The hair product of claim 40 wherein said previously frozen particles of hair are 2 mm or less in length.

50. The hair product of claim 40 said previously frozen particles of hair are 1 mm or less in length.

51. A method for thickening human hair, comprising:
harvesting particles of human hair, each of said particles ranging in all dimensions from 3 mm or less; and
applying said particles of human hair to human hair, thereby thickening human hair.

52. The method of claim 51 wherein said human hair is affixed to a person.

53. The method of claim 52 further comprising affixing said particles of human hair to human hair affixed to said person.

54. The method of claim 51 further comprising affixing said particles of hair to said human hair.

55. The method of claim 54 further comprising affixing said particles with hairspray.

56. The method of claim 54 further comprising combining said particles of hair with a carrier before affixing said particles to human hair.

57. The method of claim 56 wherein said carrier is selected from a group of carriers consisting of mousse, cream, gel, pomade, and spray.

58. The method of claim 56 wherein said carrier is a hair styling product.

59. The method of claim 51 wherein said human hair is located on human scalp.

60. The method of claim 51 wherein said human hair is located on a brow region.

61. The method of claim 51 wherein said human hair is located on a body surface.

62. The method of claim 51 further comprising harvesting said particles of human hair by cutting human hair strands.

63. The method of claim 54 wherein said human hair particles are the same color as said hair to which said human hair particles are affixed.

64. The method of claim 54 wherein said human hair particles are a color different from a color of said hair to which said human hair particles are affixed.

65. The method of claim 51 wherein said particles of human hair are powder.

66. The method of claim 51 wherein said particles of human hair are granular.

67. The method of claim 51 further comprising harvesting particles of human hair of a desired color.

68. The method of claim 51 further comprising harvesting particles of human hair of a desired type.

69. The method of claim 51 further comprising harvesting a selected dimension of particle.

70. The method of claim 69 wherein said dimension is a length.

71. The method of claim 69 wherein said dimension is a diameter.

72. The method of claim 51 wherein said particles are round.

73. The method of claim 51 further comprising separating said particles of human hair by size.

74. The method of claim 51 wherein said particles of human hair are 2 mm or less in length.

75. The method of claim 74 wherein said particles are 1 mm or less in length.

76. The method of claim 51 wherein individual particles comprise an entire structure of hair.

77. The method of claim 51 wherein said particles of hair comprise a mixture containing all elements of an entire structure of hair.

78. The method of claim 51 wherein harvesting said particles occurs by milling hair strands larger than said particles without freezing said hair strands.

79. The method of claim 77 wherein said mill is an ultra-centrifugal mill.

80. The method of claim 77 wherein said mill is a planetary ball mill.

81. The method of claim 77 further comprising washing said particles of human hair after milling.

82. The method of claim 77 further comprising imparting a fragrance to said particles of hair after milling.

83. The method of claim 77 further comprising airing out said particles of hair after milling.
